(19) United States
(12) Patent Application Publication
(10) Pub. No.: US 2005/0159659 A1
(43) Pub. Date: Jul. 21, 2005

(54) Catheter for transdiaphragmatic pressure and diaphragm electromyogram recording using helicoidal electrodes

(76) Inventors: Mohamad Sawan, Laval (CA); Francois Bellemare, Longueuil (CA); Jerome Dido, Paris (FR); Tommy Desilets, Montreal (CA)

Correspondence Address: TESTA, HURWITZ & THIBEAULT, LLP, HIGH STREET TOWER, 125 HIGH STREET, BOSTON, MA, 02110, US

(21) Appl. No.: 10/759,375

(22) Filed: Jan. 16, 2004

(51) Int. Cl. A61B 5/04; A61B 5/103; A61B 5/117

(52) U.S. Cl. 600/380; 600/593; 600/546

(57) ABSTRACT

A first aspect of the present invention is concerned with a double-helix electrode structure for sensing electrical activity of a patient's diaphragm, comprising first and second helical electrodes disposed in a double-helix arrangement for being positioned in the gastro-esophageal sphincter of the patient's diaphragm in view of sensing electrical activity of the patient's diaphragm. According to a second aspect, the present invention provides a pressure detection and acquisition device comprising a semiconductor substrate, a pressure sensor implemented on the semiconductor substrate and producing, when subjected to an external pressure, a pressure representative signal, and a signal acquisition and transmission circuit integrated to the semiconductor substrate, connected to the pressure sensor, and supplied with the pressure representative signal. Other aspects of the present invention relate to an EMG_{di} signal and pressure acquisition catheter.

### FIELD OF THE INVENTION

The present invention relates to a double-helix electrode structure for sensing electrical activity of the diaphragm of a patient, a pressure detection and acquisition device, and an EMG_{di} signal and pressure acquisition catheter.

### BACKGROUND OF THE INVENTION

Measurement of the electrical activity of the respiratory muscles (EMG) is an efficient method for representing the activity of the respiratory centers independently of the mechanical properties of the patient's respiratory system and the muscles themselves. The diaphragm EMG (EMG_{di}) can be measured through an esophageal electrode structure. EMG_{di} recording is particularly useful since the diaphragm is the principal respiratory muscle of the human being and the postural contribution of the diaphragm is much less important than that of the thoracic and abdominal muscles. Accordingly, electrical activity of the diaphragm is closely related to the activation of the respiratory centers.

Joint knowledge of the EMG_{di} and trans-diaphragmatic pressure can be used to evaluate the electromechanical coupling of the diaphragm (trans-diaphragmatic pressure/EMG_{di}), which is very useful to diagnose muscular-related pathologies. However, the complexity of installation of the different components required for this kind of measurements and the difficulty of analyzing the resulting signals impede clinical use of these data; these data are acquired and used only in the context of research.

Acquisition of the EMG_{di} through the esophageal path has been traditionally conducted by positioning electrodes at the level of the gastro-esophageal sphincter, i.e. at the location where the esophagus passes through the diaphragm [Luo, Y. M. et al. (1999); “Quantification of the esophageal diaphragm electromyogram with magnetic phrenic nerve stimulation”; American Journal of respiratory and critical care medicine; 160; 1629-1634]. For that purpose, an esophageal catheter bearing electrodes is introduced through one nostril or the mouth of the patient, and the electrodes are positioned by trial and error at the level of the gastro-esophageal sphincter. In the past, pairs of bipolar electrodes in a series of equidistant annular electrodes have been used. The EMG_{di} corresponds to the difference of potential between the annular electrodes of one pair of the series.

In practice, EMG_{di} signals are contaminated by ECG whose spectrum overlaps the spectrum of the EMG_{di} as well as by filtering effects due to the position of the innervation centers about the annular electrodes. To attenuate the contamination of the EMG_{di} by the ECG, complex “subtraction” algorithms [Levine, S. et al. 1986. “Description and validation of an ECG removal procedure for EMG_{di} power spectrum analysis”. Journal of Applied Physiology, Vol. 60(3): 1073 -1081.] or simpler “masking” algorithms of unproven efficiency [Schweitzer, T. et al. 1979. “Spectral analysis of human respiratory diaphragmatic electromyograms”; Journal of Applied Physiology: Respiratory Environmental and Exercise Physiology; Vol. 46(1); 152-165] have been used.

Since the acquired analog EMG_{di} signals are conveyed outside the patient through wires running along the esophageal catheter, these electrical wires act as an antenna to collect further contamination signals, for example a 60 Hz signal from the electrical mains. Metallic shielding of the wires is not always sufficient to eliminate this problem.

Also, longitudinal positioning of the esophageal catheter is a source of problems. A study [Beck, J. et al. 1997; “Diaphragm interference pattern EMG and compound muscle action potentials: effects of chest wall configuration”; Journal of applied physiology; 82 : 2; 520-530] has demonstrated that the RMS (Root Mean Square) amplitude value and the central frequency of the power spectrum are affected by the position of the catheter-mounted electrodes with respect to the innervation zone of the diaphragm. Although it is possible to correctly position, by trial and error or through the use of more or less complex algorithms, the series of annular catheter-mounted electrodes, movements of the diaphragm still induce unavoidable artifacts that highly complicate signal analysis.

Regarding the trans-diaphragmatic pressure, i.e. the difference between the patient's gastric and esophageal pressures, it is conventionally measured through balloons about 10 centimeters long and connected to external pressure sensors. A variant used in pediatrics makes use of water coupling. The above systems are efficient but hinder the patients, and present important drawbacks such as leak problems or bad frequency responses. Other methods based on micro-electromechanical or optical pressure sensors are presently under study, but clinical use thereof is still rare [[Chartrand, D. A., Jodoin, C. et Couture, J; (1991). “Measurement of pleural pressure with esophageal catheter-tip micro-manometer in anaesthetized humans”; Canadian Journal of Anaesthesia; 38; 518-521] [Gilbert, R. et al.; (1979); “Measurement of transdiaphragmatic pressure with a single gastric-esophageal probe”; Journal of Applied Physiology; 47; 628-630] [Hodges, P. W. and Gandevia, S. C.; (2000); <<Changes in intra-abdominal pressure during postural and respiratory activation of the human diaphragm>>; Journal of Applied Physiology; 89: 967-976]].

### SUMMARY OF THE INVENTION

According to a first aspect of the present invention, there is provided a double-helix electrode structure for sensing electrical activity of the diaphragm of a patient, comprising first and second helical electrodes disposed in a double-helix arrangement for being positioned in the gastro-esophageal sphincter of the patient's diaphragm in view of sensing electrical activity of the patient's diaphragm.

According to a second aspect of the present invention, there is provided a pressure detection and acquisition device, comprising a semiconductor substrate, a pressure sensor implemented on the semiconductor substrate, and a signal acquisition and transmission circuit. The pressure sensor produces, when subjected to an external pressure, a pressure representative signal. The signal acquisition and transmission circuit is integrated to the semiconductor substrate, is connected to the pressure sensor, and is supplied with the pressure representative signal.

According to a third aspect of the invention, there is also provided an EMG_{di} signal and pressure acquisition catheter, comprising an esophageal catheter, an EMG_{di} signal detection electrode structure, a gastric pressure sensor, an esophageal pressure sensor, and an acquisition and transmission circuit. The esophageal catheter has an EMG_{di} signal and pressure acquisition portion, and the EMG_{di} signal detection electrode structure is mounted on the acquisition portion of the esophageal catheter to detect an EMG_{di} signal produced by the diaphragm of a patient. The gastric pressure sensor is mounted on the acquisition portion of the esophageal catheter on a first side of the EMG_{di} signal detection electrode structure, to detect gastric pressure of the patient. The esophageal pressure sensor is mounted on the acquisition portion of the esophageal catheter on a second side of the EMG_{di} signal detection electrode structure opposite to the first side, to detect esophageal pressure of the patient. Finally, the acquisition and transmission circuit is mounted on the acquisition portion of the esophageal catheter, is connected to the EMG_{di} signal detection electrode structure, the gastric pressure sensor and the oesophageal pressure sensor, and is supplied with the detected EMG_{di} signal, the detected gastric pressure and the detected esophageal pressure.

The foregoing and other objects, advantages and features of the present invention will become more apparent upon reading of the following non-restrictive description of illustrative embodiments thereof, given by way of example only with reference to the accompanying drawings.

### BRIEF DESCRIPTION OF THE DRAWINGS

In the appended drawings:

FIG. 1 is a schematic view of an illustrative embodiment of a system according to the present invention for the simultaneous recording of both a patient's EMG_{di} and trans-diaphragmatic pressure;

FIG. 2a is a side elevational view of an illustrative embodiment of a one-turn double-helix electrode structure according to the present invention for sensing a patient's EMG_{di};

FIG. 2b is a side elevational view of an illustrative embodiment of a two-turn double-helix electrode structure according to the present invention for sensing a patient's EMG_{di};

FIG. 3 is a perspective view of an example of EMG_{di} electrode comprising a linear array of annular electrodes;

FIG. 4 is a graph showing the efficiency of the one-turn and two-turn double-helix electrode structures of FIGS. 2a and 2b to reduce ECG contamination in the EMG_{di} signal in comparison with the linear array of annular electrodes of FIG. 3 taken as reference electrode structure;

FIG. 5a is a first example of micro-electromechanical pressure sensor integrated to a semiconductor substrate;

FIG. 5b is a second example of micro-electromechanical pressure sensor integrated to a semiconductor substrate;

FIG. 5c is a third example of micro-electromechanical pressure sensor integrated to a semiconductor substrate;

FIG. 5d is a fourth example of micro-electromechanical pressure sensor integrated to a semiconductor substrate;

FIG. 5e is a fifth example of micro-electromechanical pressure sensor integrated to a semiconductor substrate;

FIG. 5f is a sixth example of micro-electromechanical pressure sensor integrated to a semiconductor substrate;

FIG. 5g is a seventh example of micro-electromechanical pressure sensor integrated to a semiconductor substrate;

FIG. 6 is a top plan view of a layout of piezoelectric elements mounted on the top face of the pressure-deformable membrane;

FIG. 7 is a Wheatstone bridge circuit in which the piezoelectric elements of FIG. 6 are connected;

FIG. 8 is a schematic view of an illustrative embodiment of a pressure detection and acquisition device according to the invention, comprising a pressure sensor and a portion of a signal acquisition and transmission circuit both integrated on a same semiconductor substrate; and

FIG. 9 is a schematic block diagram of an illustrative embodiment of first and second portions of the signal acquisition and transmission circuit according to the present invention.

### DETAILED DESCRIPTION OF THE ILLUSTRATIVE EMBODIMENTS

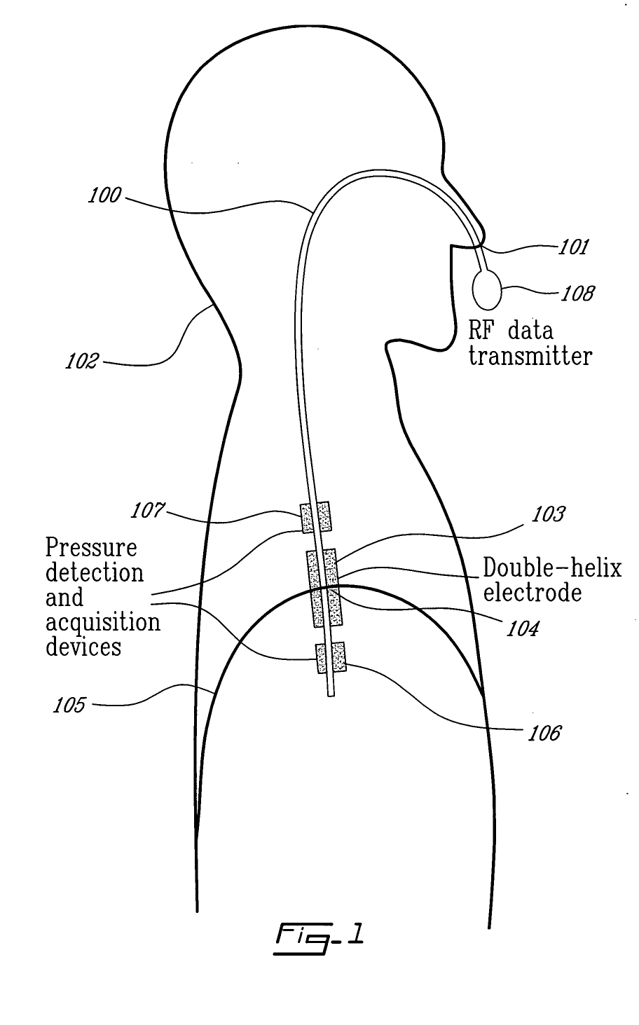

As illustrated in FIG. 1, according to a non-restrictive illustrative embodiment of the present invention, there is provided a system for the simultaneous recording of both a patient's EMG_{di} and trans-diaphragmatic pressure. For that purpose, the system of FIG. 1 comprises the following components:
an esophageal catheter 100 to be introduced through a nostril 101 or the mouth of a patient 102;
a double-helix electrode structure 103 mounted on the esophageal catheter 100 and to be positioned in the gastro-esophageal sphincter 104 of the patient's diaphragm 105;
a first pressure detection and acquisition device 106 for acquiring, analog-to-digital converting and serially transmitting both the patient's gastric pressure and EMG_{di} from the double-helix electrode 103, this first pressure detection and acquisition device 106 incorporating a gastric pressure sensor; and
a second pressure detection and acquisition device 107 for acquiring and analog-to-digital converting the patient's esophageal pressure, for receiving the gastric pressure and EMG_{di} serially transmitted from the first pressure detection and acquisition device 106, and serially transmitting data related to the patient's esophageal and gastric pressures, and the EMG_{di} toward a central data processing system (not shown), this second pressure detection and acquisition device 107 incorporating an esophageal pressure sensor.

As illustrated in FIG. 1, the first pressure detection and acquisition device 106 and its associated gastric pressure sensor and the second pressure detection and acquisition device 107 and its associated esophageal pressure sensor are mounted on the esophageal catheter 100 on opposite sides of the double-helix electrode structure 103 and, therefore, on opposite sides of the patient's diaphragm 105. Obviously, the gastric pressure sensor the first pressure detection and acquisition device 106 and its associated gastric pressure sensor and the second pressure detection and acquisition device 107 and its associated esophageal pressure sensor are mounted on the esophageal catheter 100 on opposite sides of the double-helix electrode structure 103 and, therefore, on opposite sides of the patient's diaphragm 105.

The system of FIG. 1 enables in situ measurement of a patient's EMG_{di} and gastric and esophageal pressures, as well as acquisition, analog-to-digital conversion and transmission of these data toward a central signal and data processing system (not shown).

### Illustrative Embodiment of the Double-Helix EMG_{di} Electrode

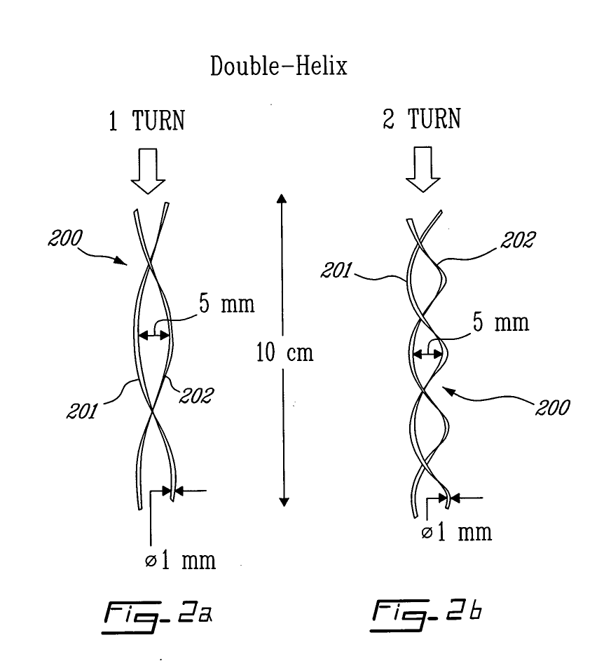

Referring to FIGS. 2a and 2b, the illustrative embodiment of double-helix EMG_{di} electrode structure 200 according to the present invention has a double-helix geometry similar to the structure of a DNA molecule. To produce this double-helix electrode structure, two electrically conductive straight electrodes are wound on themselves to implement the double helix geometry.

More specifically, the double-helix electrode structure 200 comprises first 201 and second 202 helical electrodes disposed in a double-helix arrangement for being positioned in the gastro-oesophageal sphincter 104 of the patient's diaphragm 105. This double-helix arrangement comprises a longitudinal, geometrical axis (not shown) and constitutes a symmetrical arrangement of helical electrodes 201 and 202 about this longitudinal, geometrical axis. The first and second helical electrodes 201 and 202 are therefore coaxial electrodes highly symmetrical about the longitudinal, geometrical axis.

The double-helix geometry presents the advantage of filtering signals propagating from radially remote sources, for example ECG, while preserving signals from closer sources, for example the muscular fibers of the patient's diaphragm near the gastro-esophageal sphincter. Since the double-helix geometry forms a highly symmetrical structure, contamination from ECG or any other remote sources appears with substantially the same amplitude on both helical electrodes 201 and 202 and is, if not completely eliminated, substantially reduced when the signals on these twin electrodes are differentially amplified. In this manner, contamination of the EMG_{di} signal by ECG or any other remote sources is, if not completely eliminated, substantially reduced.

Those of ordinary skill in the art will appreciate that the efficiency of the double-helix geometry of the electrode structure 200 can be improved by appropriately adjusting geometrical parameters such as the number of turns of the helical electrodes 201 and 202, the nature of the material used to fabricate the electrodes 201 and 202, the pitch and length of the helical electrodes 201 and 202, the diameter of the helical electrodes 201 and 202, etc.

For example, each helical electrode will comprise at least one turn. A non-restrictive range of number of turns could for example be between 1 and 4.

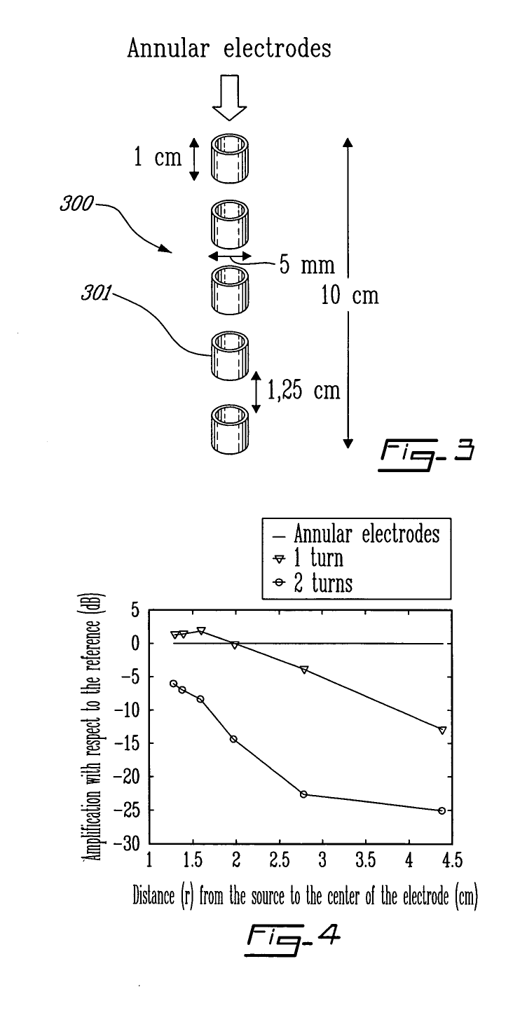

Tests have been conducted to compare the efficiency of the double-helix electrode structure 200 with respect to a traditional, linear array 300 of annular, cylindrical electrodes such as 301 (FIG. 3). These tests have confirmed that the double-helix electrode structure 200 substantially reduces the ECG contamination.

FIG. 4 is a graph showing the effect of the radial distance r between a punctual signal source and the EMG_{di} electrode structure on the difference of potential between these electrodes, for the one-turn double-helix electrode structure or FIG. 2a, the two-turn double-helix electrode structure of FIG. 2b, and the electrode array 300 of FIG. 3 including a series of five (5) annular electrodes 301 and mounted, for example on an esophageal catheter.

In FIG. 4, the array 300 of annular electrodes 301 is taken as a reference. Therefore, the 0 dB axis of the graph of FIG. 4 corresponds to the reference electrode array 300 including a series of five (5) annular electrodes 301.

The electrode structures 200 and electrode array 300 have similar overall dimensions to facilitate their comparison. As illustrated in FIG. 3, each annular electrode 301 of the reference electrode array 300 is an electrically conductive cylinder having a length of about 1 cm and a diameter of about 5 mm. The spacing between two consecutive annular electrodes 301 is about 1.25 cm and the global length of the electrode array 300 is about 10 cm. The individual helical electrodes 201 and 202 of the one-turn and two-turn double-helix electrode structures 200 of FIGS. 2a and 2b are made of an electrically conductive wire having a diameter equal to about 1 mm. Again, the one-turn and two-turn double-helix electrode structures of FIGS. 2a and 2b both have a global diameter of about 5 mm and a global length of about 10 cm.

The curves of FIG. 4 clearly show that the double-helix electrode structures 200 of FIGS. 2a and 2b present a filtering effect much more pronounced than that of the reference electrode array 300 of FIG. 3. For example, the curves of FIG. 4 show that the one-turn double-helix electrode structure 200 of FIG. 2a will attenuate a punctual signal source located at a radial distance of 4 cm from the axis of the electrode structure by about 10 dB with respect to the reference electrode array 300. In the same manner, the curves of FIG. 4 show that the two-turn double-helix electrode structure 200 of FIG. 2b will attenuate a punctual signal source located at a radial distance of 4 cm from the axis of the electrode structure by about 25 dB with respect to the reference electrode array 300. The distance of 4 cm substantially corresponds to the distance between the gastro-esophageal sphincter and the heart. It can be concluded that the level of attenuation varies with the pitch of the helical electrodes 201 and 202 and the number of turns. FIG. 4 accordingly shows that the two-turn double-helix electrode structure of FIG. 2b is more efficient than the one-turn double-helix electrode structure of FIG. 2a in damping ECG contamination.

### Illustrative Embodiment of the Pressure Detection and Acquisition Devices

As illustrated in FIG. 1, the first and second pressure detection and acquisition device 106 and 107 are mounted on the esophageal catheter 100 on opposite sides of the double-helix electrode structure 103 and, therefore, on opposite sides of the patient's diaphragm 105, and each comprise a signal acquisition and transmission circuit and a pressure sensor.

### Illustrative Embodiment of the Pressure Sensor

According to this illustrative embodiment, the gastric and esophageal pressure sensors are micro-electromechanical pressure sensors. Micro-electromechanical pressure sensors present the advantage of offering a performance comparable to that of latex balloons while presenting a small volume and low cost of fabrication. They can also be integrated, along with the corresponding portion of the signal acquisition and transmission circuit, to a common semiconductor substrate.

In view of reducing as much as possible the overall external dimensions, a “monolithic” approach is used to fabricate the pressure sensor and the associated portion of the signal acquisition and transmission circuit on the same semiconductor substrate, in particular but not exclusively a silicon substrate. The monolithic approach also improves the precision of construction of the pressure sensor. However, it should be kept in mind that it is within the scope of the present invention to use other approaches to manufacture the pressure sensors, for example an “hybrid” approach in which the pressure sensor is manufactured separately and subsequently assembled to the semiconductor substrate bearing the corresponding portion of the signal acquisition and transmission circuit, using for example techniques such as “flip-chip” or “wire bonds”. This interconnection will, however, reduce the precision of construction of the pressure sensors.

Micro-electromechanical pressure sensors comprise a membrane deformable by pressure. Capacitive or piezoelectric elements are mounted on this membrane to convert the deformation to an electric, pressure representative signal.

Capacitive pressure sensors generally comprise two electrically conducting planar surfaces, including a fixed surface and a movable surface on the pressure-deformable membrane. These electrically conducting surfaces form a capacitor having a variable capacitance, for example, inversely proportional to the applied pressure. Capacitive pressure sensors present a high accuracy and a low sensitivity to temperature. However, they require relatively large surfaces.

Piezoelectric pressure sensors comprise resistive zones or elements deposited or implanted on the pressure-deformable membrane. When the membrane deforms in response to an external pressure, the resistance value of the piezoelectric zones or elements changes. This change in resistance value can be easily detected through a simple detector circuit, for example a Wheatstone bridge.

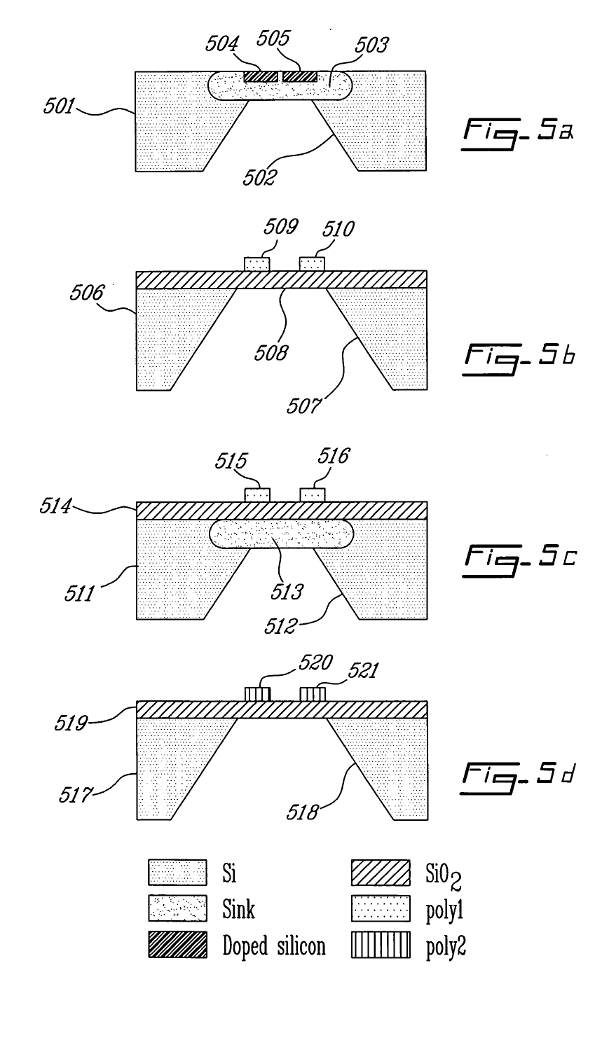

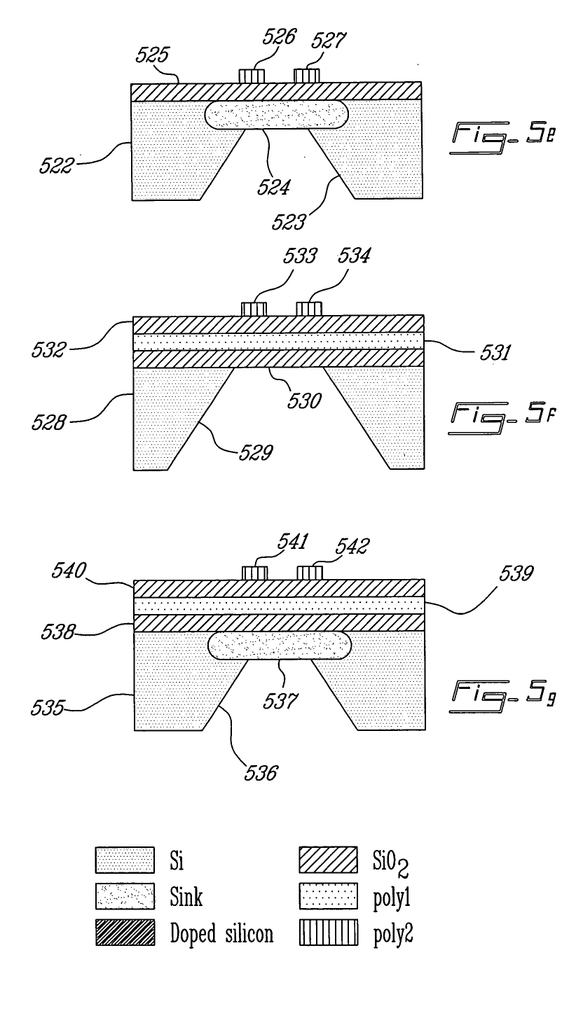

Examples of micro-electromechanical piezoelectric pressure sensors integrated to a silicon substrate are illustrated in FIG. 5a-5g.

In FIG. 5a, the silicon substrate 501 is formed with a square, tapering cavity 502 defining a square opening covered by the pressure-deformable membrane 503. The pressure-deformable membrane 503 is made of a sink-P layer formed by an implantation of Boron ions diffused 3 μm deep within the silicon substrate 501. The piezoelectric elements 504 and 505 are made of p⁺-doped silicon (Si) regions formed substantially in the center of the top face of the pressure-deformable membrane 503. Deformation of the membrane 503 by the application of an external pressure will change the resistance values of the piezoelectric elements 504 and 505, and this variation of resistance value will be detected to produce a pressure-representative signal.

In FIG. 5b, the silicon substrate 506 is formed with a square, tapering cavity 507 defining a square opening. The pressure-deformable membrane 508 is made of a SiO₂ layer covering a portion of the silicon substrate 506 including the square opening. The piezoelectric elements 509 and 510 are made of poly-silicon 1; poly-silicon 1 is a 0.3 μm thick deposit of polycrystalline silicon deposited by Low Pressure Chemical Vapor Deposition (LPCVD) and shaped by etching substantially in the center of the top face of the pressure-deformable membrane 508. Deformation of the pressure-deformable membrane 508 by the application of an external pressure will change the resistance value of the piezoelectric elements 509 and 510, and this variation of resistance value will be detected to produce a pressure-representative signal.

In FIG. 5c, the silicon substrate 511 is formed with a square, tapering cavity 512 defining a square opening covered by the pressure-deformable membrane. The pressure-deformable membrane is made of:
a sink-P layer 513 formed by an implantation of Boron ions diffused 3 μm deep within the silicon substrate 511; and
a SiO₂ layer 514 covering a portion of the silicon substrate 511 including the sink-P layer 513;
   The piezoelectric elements 515 and 516 are made of poly-silicon 1; poly-silicon 1 is a 0.3 μm thick deposit of polycrystalline silicon deposited by Low Pressure Chemical Vapor Deposition (LPCVD) and shaped by etching substantially in the center of the top face of the pressure-deformable membrane 513-514. Deformation of the pressure-deformable membrane 513-514 by the application of an external pressure will change the resistance value of the piezoelectric elements 515 and 516, and this variation of resistance value will be detected to produce a pressure-representative signal.

In FIG. 5d, the silicon substrate 517 is formed with a square, tapering cavity 518 defining a square opening. The pressure-deformable membrane 519 is made of a SiO₂ layer covering a portion of the silicon substrate 517 including the square opening. The piezoelectric elements 520 and 521 are made of poly-silicon 2; poly-silicon 2 is a 0.3 μm thick deposit of polycrystalline silicon shaped by etching substantially in the center of the top face of the pressure-deformable membrane 519. Deformation of the pressure-deformable membrane 519 by the application of an external pressure will change the resistance value of the piezoelectric elements 520 and 521 and this variation of resistance value will be detected to produce a pressure-representative signal.

In FIG. 5e, the silicon substrate 522 is formed with a square, tapering cavity 523 defining a square opening covered by the pressure-deformable membrane. The pressure-deformable membrane is made of:
a sink-P layer 524 formed by an implantation of Boron ions diffused 3 μm deep within the silicon substrate 522; and
a SiO₂ layer 525 covering a portion of the silicon substrate 522 including the sink-P layer 524;
   The piezoelectric elements 526 and 527 are made of poly-silicon 2; poly-silicon 2 is a 0.3 μm thick deposit of polycrystalline silicon shaped by etching substantially in the center of the top face of the pressure-deformable membrane 524-525. Deformation of the pressure-deformable membrane 524-525 by the application of an external pressure will change the resistance value of the piezoelectric elements 526 and 527, and this variation of resistance value will be detected to produce a pressure-representative signal.

In FIG. 5f, the silicon substrate 528 is formed with a square, tapering cavity 529 defining a square opening covered by the pressure-deformable membrane. The pressure-deformable membrane is made of:
a SiO₂ layer 530 covering a portion of the silicon substrate 528 including the square opening;
a layer 531 of poly-silicon 1 on top of the SiO₂ layer 530; poly-silicon 1 is a 0.3 μm thick deposit of polycrystalline silicon deposited by Low Pressure Chemical Vapor Deposition (LPCVD); and
a SiO₂ layer 532 covering the layer 531 of poly-silicon 1.
   The piezoelectric elements 533 and 534 are made of poly-silicon 2; poly-silicon 2 is a 0.3 μm thick deposit of polycrystalline silicon shaped by etching substantially in the center of the top face of the pressure-deformable membrane 530-532. Deformation of the pressure-deformable membrane 530-532 by the application of an external pressure will change the resistance value of the piezoelectric elements 533 and 534, and this variation of resistance value will be detected to produce a pressure-representative signal.

In FIG. 5g, the silicon substrate 535 is formed with a square, tapering cavity 536 defining a square opening covered by the pressure-deformable membrane. The pressure-deformable membrane is made of:
a sink-P layer 537 formed by an implantation of Boron ions diffused 3 μm deep within the silicon substrate 535;
a SiO₂ layer 538 covering a portion of the silicon substrate 535 including the sink-P layer 537;
a layer 539 of poly-silicon 1 on top of the SiO₂ layer 538; poly-silicon 1 is a 0.3 μm thick deposit of polycrystalline silicon deposited by Low Pressure Chemical Vapor Deposition (LPCVD); and
a top SiO₂ layer 540 covering the layer 539 of poly-silicon 1.

The piezoelectric elements 541 and 542 are made of poly-silicon 2; poly-silicon 2 is a 0.3 μm thick deposit of polycrystalline silicon shaped by etching substantially in the center of the top face of the pressure-deformable membrane 537-540. Deformation of the pressure-deformable membrane 537-540 by the application of an external pressure will change the resistance value of the piezoelectric elements 541 and 542, and this variation of resistance value will be detected to produce a pressure-representative signal.

For the sake of simplicity, the usual top oxide layers have been voluntarily omitted from FIGS. 5a-5g.

Since solidity of the pressure-deformable membrane will increase with thickness thereof, the solutions of FIGS. 5f and 5g appears “a priori” more convenient than those of FIGS. 5a-5e, taking into consideration the levels of pressure to be measured.

This is believed to be within the knowledge of one of ordinary skill in the art to design a process of manufacture of the micro-electromechanical pressure sensors of FIGS. 5a-5g. Such processes of manufacture forms no part of the present invention and, accordingly, will not be further described in the present specification.

It is also within the scope of the present invention to use another type of pressure sensors, micro-electromechanical or not, integrated or not to the semiconductor substrate, fabricated according to the same or different processes, as long as the pressure sensor can be mounted on the semiconductor substrate itself subsequently mounted on the esophageal catheter 100, using similar or different materials.

In a piezoelectric zone or element, the ratio of the variation of resistance ΔR with respect to the initial resistance R₀ is given by the following relation:

$$\frac{Δ R}{{R}_{0}} = K ( {ɛ}_{⊥} + {ɛ}_{//} )$$

where ε_{⊥} and ε_{∥} are the perpendicular and parallel deformations, respectively and K is the gauge coefficient depending on the type of material and the temperature.

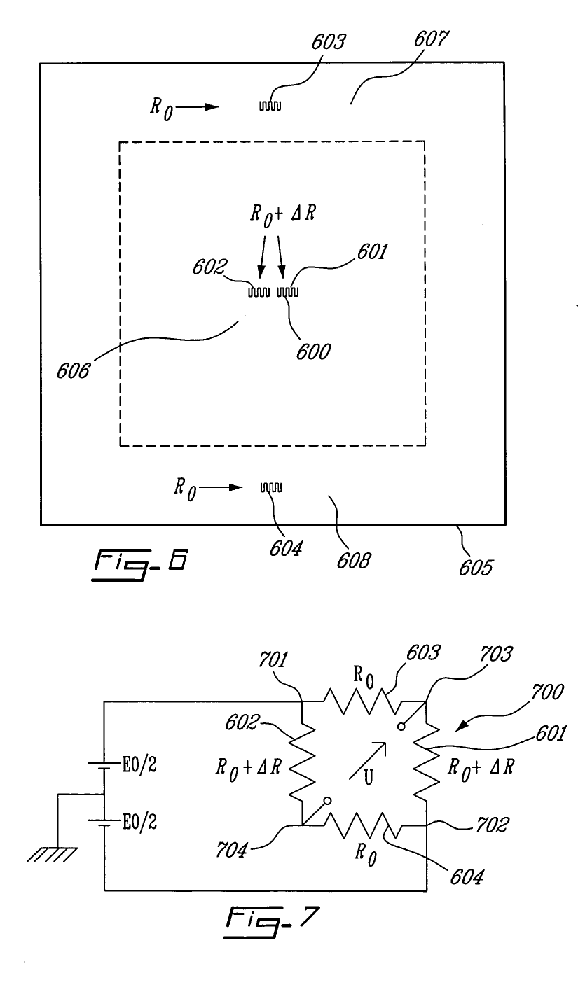

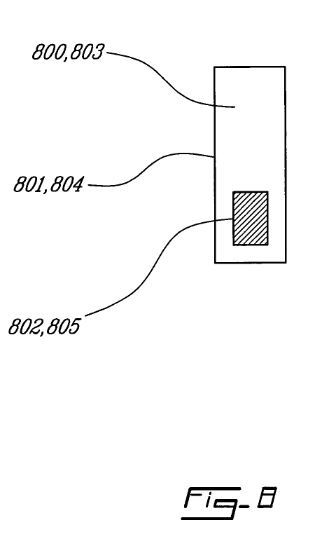

Therefore, in order to adequately measure the variation of resistance ΔR and therefore a pressure value proportional to this variation of resistance ΔR, the piezoelectric zone or element, for example in the form of a serpentine structure such as 600 in FIG. 6, can be connected in a Wheatstone bridge circuit 700 as illustrated in FIG. 7. For that purpose, four piezoelectric, resistive paths 601-604 (FIG. 6) parallel to each other are formed on the top face of the pressure-deformable membrane 605: two paths 601 and 602 substantially in the pressure-deformable center 606 of the membrane 605 and two paths 603 and 604 on opposite external sides 607 and 608, respectively, of the same membrane 605 where this membrane does not deform. Upon deformation of the membrane 605 in response to an external pressure, the central piezoelectric elements 601 and 602 deform and their resistance value passes from R₀ to R₀+ΔR. On the contrary, the side piezoelectric elements 603 and 604 are not subjected to deformation and their resistance value remains equal to R₀.

The resulting Wheatstone bridge circuit 700 is illustrated in FIG. 7. The four piezoelectric elements 601-604 whose resistance values are equal to either R₀ and R₀+ΔR, are connected in the Wheatstone bridge circuit 700 as shown in FIG. 7. An electromotive force E₀ is applied between diagonal points 701 and 702 of the Wheatstone bridge circuit 700 and the voltage U, representative of the measured pressure, is detected between diagonal points 703 and 704.

The Wheatstone bridge circuit 700 of FIG. 7 constitutes a very efficient and accurate means for measuring the level of pressure applied to the pressure-deformable membrane 605.

### Illustrative Embodiment of the Signal Acquisition and Transmission Circuit

The signal acquisition and transmission circuit has the following three functions:
acquire the analog gastric pressure signal, the EMG_{di} signal and the esophageal pressure signal;
convert the acquired analog signals to digital signals; and
transmit the digital data toward an external central data processing system (not shown); serial transmission is advantageous since it will reduce the number of wires running through the esophageal catheter 100 (FIG. 1), and accordingly the size of the esophageal catheter.

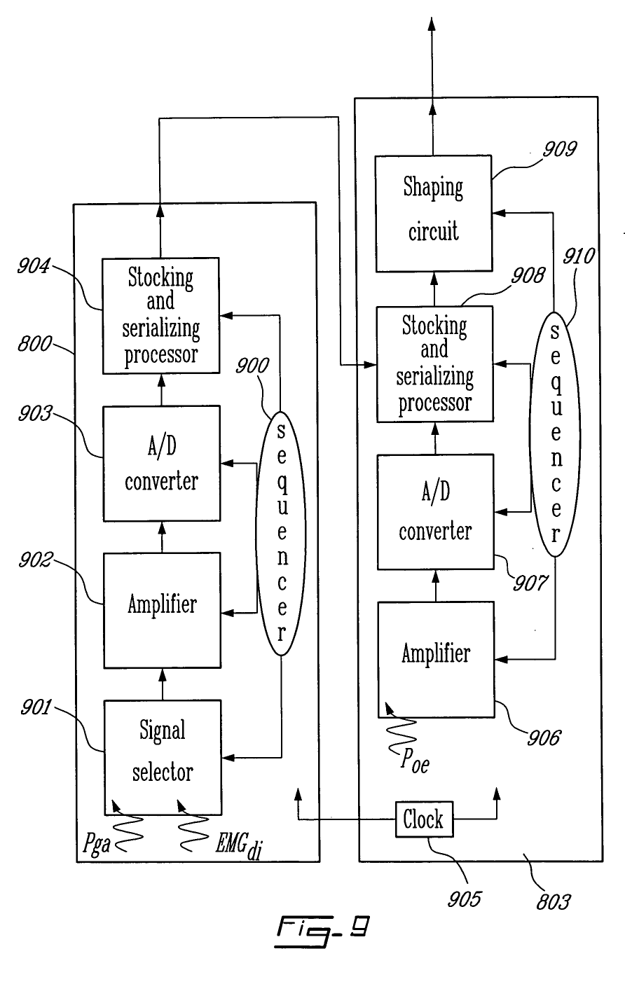

Referring to FIG. 8, a first portion 800 of the signal acquisition and transmission circuit is integrated on the same semiconductor substrate 801 as the gastric pressure sensor 802 to form the first pressure detection and acquisition device 106 (FIG. 1).

Still referring to FIG. 8, a second portion 803 of the signal acquisition and transmission circuit is integrated on the same semiconductor substrate 804 as the esophageal pressure sensor 805 to form the second pressure detection and acquisition device 107 (FIG. 1).

Referring to FIG. 9, the first portion 800 of the signal acquisition and transmission circuit first comprises a sequencer 900 for controlling the various operations of the first portion 800.

A signal selector 901 is responsive to a command from the sequencer 900 to successively select the gastric pressure signal P_{ga} or the EMG_{di} signal as input signal. Only a pair of wires, running through the catheter 100, is therefore required between the respective helical electrodes of the double-helix electrode structure 103 and the first portion 800 of the signal acquisition and transmission circuit.

Still under the control of the sequencer 900, the selected signal is then amplified by at least one amplifier 902, converted to a digital signal by at least one analog-to-digital (A/D) converter 903, and then stocked and serialized in at least one stocking and serializing processor 904. To reduce the number of wires running through the esophageal catheter 100, the resulting serial data are then transmitted from processor 904 to a stocking and serializing processor 908 of the second circuit portion 803. Therefore, only a serial transmission line is required between the stocking and serializing processor 904 and 908 of the first and second portions 800 and 803 of the signal acquisition and transmission circuit.

The signal selector 901 may simply comprise transmission electronic gates. The amplifier 902 may be a differential amplifier and the stocking and serializing processor 904 may be formed of a shift register charged synchronously in parallel or in series. The sequencer 900 may be a timer circuit for controlling the periods of operation of the different modules 901-904 of the first portion 800 of the signal acquisition and transmission circuit.

The two signals P_{ga} and EMG_{di} can be processed through a same chain of amplifier, A/D converter and stocking and serializing processor or two different chains.

The second portion 803 of the signal acquisition and transmission circuit comprises, as illustrated in FIG. 9, a clock 905 supplied to both the first 800 and second 803 portions of the acquisition and transmission circuit for timing the various operations performed by these first and second portions 800 and 803; an additional clock line can then be required between the first and second portions 800 and 803 of the signal acquisition and transmission circuit. A clock can also be provided in the two portions 800 and 803; synchronization of the two portions 800 and 803 is then required.

Still referring to FIG. 9, the second portion 803 of the signal acquisition and transmission circuit comprises a sequencer 910 for controlling the operations performed by this second portion 803.

Under the control of the sequencer 910, the esophageal gastric pressure signal Pₒₑ is amplified by an amplifier 906, converted to a digital signal by an analog-to-digital converter 907, and then stocked and serialized in processor 908. The serial data stocked in the stocking and serializing processor 908 are transferred to a shaping 909 circuit prior to transmission of these data toward the external data processing system (not shown). The shaping circuit 909 is responsible for the arrangement of the data to be transmitted according to a predetermined transmission protocol that can be recognized by the external signal and data processing system. A single serial line (not shown), running through the catheter 100 toward the proximal end thereof, is then required for transmitting the data from the shaping circuit 909.

Again, the amplifier 906 may be a differential amplifier and the stocking and serializing processor 908 may be formed of a shift register charged synchronously in parallel or in series. The sequencer 910 may be a timer circuit for controlling the periods of operation of the different modules 906-909 of the second portion 803 of the signal acquisition and transmission circuit.

The first and second portions 800 and 803 of the acquisition and transmission circuit may further comprise:
a parity check module and/or a Cyclic Redundancy Check module to verify the integrity of the transmitted data;
filter circuits for withdrawing various signal contaminations; and
a decoder of instructions from the exterior to change the configuration of the system, for example to change the calibration mode, the gains of the amplifiers, etc.

The in situ analog-to-digital conversion of the various pressure signals P_{ga} and Pₒₑ, and EMG_{di} signal presents the advantage of eliminating the problem of parasitic noise gathered by the wires conventionally used to transmit the acquired analog signals to an external processing unit. This also enables the serial combination of many signals, transmission of these signals through a single line, and recovery of the individual signals outside the patient. Finally, the external signal and data processing system can be connected directly to this single wire without the need of complex and troublesome interface devices.

According to an illustrative embodiment illustrated in FIG. 1, the serial data from the shaping circuit 909 can be transmitted to a remote data processing system (not shown) through a RF (Radio Frequency) data transmitter 108.

Obviously, the components 900-910 can be integrated on the corresponding substrate using techniques well known to those of ordinary skill in the art. These techniques will not be further described in the present specification.

Although the present invention has been described hereinabove by way of non-restrictive illustrative embodiments thereof, these embodiments can be modified at will, within the scope of the appended claims, without departing from the spirit and nature of the present invention.

1. A double-helix electrode structure for sensing electrical activity of the diaphragm of a patient, comprising first and second helical electrodes disposed in a double-helix arrangement for being positioned in the gastro-oesophageal sphincter of the patient's diaphragm in view of sensing electrical activity of the patient's diaphragm.

2. A double-helix electrode structure as defined in claim 1, wherein the double-helix arrangement comprises a geometrical axis and constitutes a symmetrical arrangement of electrodes about said geometrical axis.

3. A double-helix electrode structure as defined in claim 1, wherein the first and second helical electrodes are coaxial electrodes.

4. A double-helix electrode structure as defined in claim 1, wherein the first and second helical electrodes each comprise at least one turn.

5. A double-helix electrode structure as defined in claim 1, wherein the double-helix electrode structure is a double-helix electrode structure having a diameter of about 5 mm and a length of about 10 cm.

6. A double-helix electrode structure as defined in claim 1, wherein the double-helix electrode structure is a double-helix electrode structure having a diameter of about 5 mm and a length of about 10 cm, and comprising helical electrodes each having a number of turns between 1 and 4.

7. A double-helix electrode structure as defined in claim 1, wherein the first and second electrodes have an ECG-attenuating pitch.

8. A double-helix electrode structure as defined in claim 1, wherein the double-helix arrangement comprises means for attenuating ECG disturbance by 10 to 25 dB with respect to an electrode structure formed of a serial array of electrodes.

9. A pressure detection and acquisition device, comprising:
a semiconductor substrate;
a pressure sensor implemented on the semiconductor substrate, said pressure sensor producing, when subjected to an external pressure, a pressure representative signal; and
a signal acquisition and transmission circuit integrated to the semiconductor substrate, said signal acquisition and transmission circuit being connected to the pressure sensor and supplied with the pressure representative signal.

10. A pressure detection and acquisition device as defined in claim 9, wherein the pressure sensor is integrated to the semiconductor substrate.

11. A pressure detection and acquisition device as defined in claim 9, wherein the pressure sensor comprises a pressure-deformable membrane having a face on which at least one piezoelectric element is mounted.

12. A pressure detection and acquisition device as defined in claim 11, wherein the pressure-deformable membrane is a semiconductor membrane and the at least one piezoelectric element is deposited on the face of the semiconductor membrane.

13. A pressure detection and acquisition device as defined in claim 11, wherein the pressure-deformable membrane is a semiconductor membrane and the at least one piezoelectric element is implanted in the semiconductor membrane.

14. A pressure detection and acquisition device as defined in claim 9, wherein the pressure detection and acquisition device is a monolithic semiconductor device.

15. A pressure detection and acquisition device as defined in claim 11, wherein the pressure-deformable membrane is made of a material selected from the group consisting of: a sink-P material formed by an implantation of Boron ions within a silicon substrate, SiO₂ and polycrystalline silicon.

16. A pressure detection and acquisition device as defined in claim 11, wherein the pressure-deformable membrane is a multi-layer membrane, and wherein each layer of the multi-layer membrane is made of a material selected from the group consisting of: a sink-P material formed by an implantation of Boron ions within a silicon substrate, SiO₂, and polycrystalline silicon.

17. A pressure detection and acquisition device as defined in claim 9, wherein the pressure-deformable membrane is made of semiconductor material and said at least one piezoelectric element is made of a material selected from the group consisting of: p⁺-doped silicon and polycrystalline silicon.

18. A pressure detection and acquisition device as defined in claim 9, wherein the signal acquisition and transmission circuit comprises, integrated to the semiconductor substrate, an amplifier for amplifying the pressure representative signal, an analog-to-digital converter for converting the amplified pressure representative signal to a digital amplified pressure representative signal, and a stocking and serializing processor supplied with the digital amplified pressure representative signal, and a sequencer for controlling operation of the amplifier, analog-to-digital converter, and stocking and serializing processor.

19. An EMG_{di} signal and pressure acquisition catheter, comprising:
an esophageal catheter having an EMG_{di} signal and pressure acquisition portion;
a EMG_{di} signal detection electrode structure mounted on the acquisition portion of the esophageal catheter to detect an EMG_{di} signal produced by the diaphragm of a patient;
a gastric pressure sensor mounted on the acquisition portion of the esophageal catheter on a first side of the EMG_{di} signal detection electrode structure, to detect gastric pressure of the patient;
an esophageal pressure sensor mounted on the acquisition portion of the esophageal catheter on a second side of the EMG_{di} signal detection electrode structure opposite to said first side, to detect esophageal pressure of the patient; and
an acquisition and transmission circuit connected to the EMG_{di} signal detection electrode structure, the gastric pressure sensor and the esophageal pressure sensor, and supplied with the detected EMG_{di} signal, the detected gastric pressure and the detected esophageal pressure.

20. An EMG_{di} signal and pressure acquisition catheter as defined in claim 19, wherein the EMG_{di} signal detection electrode structure comprises a double-helix electrode structure comprising first and second helical electrodes disposed in a double-helix arrangement for being mounted on the acquisition portion of the esophageal catheter and positioned in the gastro-esophageal sphincter of the patient's diaphragm.

21. An EMG_{di} signal and pressure acquisition catheter as defined in claim 20, wherein the double-helix arrangement comprises a geometrical axis and constitutes a symmetrical arrangement of electrodes about said geometrical axis.

22. An EMG_{di} signal and pressure acquisition catheter as defined in claim 20, wherein the first and second helical electrodes each comprise at least one turn.

23. An EMG_{di} signal and pressure acquisition catheter as defined in claim 20, wherein the double-helix arrangement comprises means for attenuating ECG disturbance.

24. An EMG_{di} signal and pressure acquisition catheter as defined in claim 19, comprising:
a first pressure detection and acquisition device, comprising:
a first semiconductor substrate;
the gastric pressure sensor implemented on the first semiconductor substrate, said gastric pressure sensor producing, when subjected to gastric pressure, a gastric pressure representative signal; and
a first portion of the acquisition and transmission circuit integrated to the first semiconductor substrate, said first portion of the acquisition and transmission circuit being connected to the gastric pressure sensor and supplied with the gastric pressure representative signal; and
a second pressure detection and acquisition device, comprising:
a second semiconductor substrate;
the esophageal pressure sensor implemented on the second semiconductor substrate, said esophageal pressure sensor producing, when subjected to esophageal pressure, an esophageal pressure representative signal; and
a second portion of the acquisition and transmission circuit integrated to the second semiconductor substrate, said second portion of the acquisition and transmission circuit being connected to the esophageal pressure sensor and supplied with the esophageal pressure representative signal.

25. An EMG_{di} signal and pressure acquisition catheter as defined in claim 24, wherein the gastric pressure sensor is integrated to the first semiconductor substrate, and the esophageal pressure sensor is integrated to the second semiconductor substrate.

26. An EMG_{di} signal and pressure acquisition catheter as defined in claim 24, wherein the first portion of the acquisition and transmission circuit is also supplied with the detected EMG_{di} signal.

27. An EMG_{di} signal and pressure acquisition catheter as defined in claim 19, wherein at least one of the gastric and esophageal pressure sensors comprises a pressure-deformable membrane having a face on which at least one piezoelectric element is mounted.

28. An EMG_{di} signal and pressure acquisition catheter as defined in claim 24, wherein the first portion of the acquisition and transmission circuit comprises, integrated to the first semiconductor substrate, an amplifier for amplifying the gastric pressure representative signal, an analog-to-digital converter for converting the amplified gastric pressure representative signal to a digital amplified gastric pressure representative signal, a stocking and serializing processor supplied with the digital amplified gastric pressure representative signal, and a sequencer for controlling operation of the amplifier, analog-to-digital converter, and stocking and serializing processor.

29. An EMG_{di} signal and pressure acquisition catheter as defined in claim 24, wherein the second portion of the acquisition and transmission circuit comprises, integrated to the second semiconductor substrate, an amplifier for amplifying the esophageal pressure representative signal, an analog-to-digital converter for converting the amplified esophageal pressure representative signal to a digital amplified esophageal pressure representative signal, a stocking and serializing processor supplied with the digital amplified esophageal pressure representative signal, and a sequencer for controlling operation of the amplifier, analog-to-digital converter, and stocking and serializing processor.

30. An EMG_{di} signal and pressure acquisition catheter as defined in claim 24, wherein:
the first portion of the acquisition and transmission circuit comprises, integrated to the first semiconductor substrate, a first amplifier for amplifying the gastric pressure representative signal, a first analog-to-digital converter for converting the amplified gastric pressure representative signal to a digital amplified gastric pressure representative signal, a first stocking and serializing processor supplied with the digital amplified gastric pressure representative signal, and a first sequencer for controlling operation of the first amplifier, first analog-to-digital converter, and first stocking and serializing processor; and
the second portion of the acquisition and transmission circuit comprises, integrated to the second semiconductor substrate, a second amplifier for amplifying the esophageal pressure representative signal, a second analog-to-digital converter for converting the amplified esophageal pressure representative signal to a digital amplified esophageal pressure representative signal, and a second stocking and serializing processor supplied with the digital amplified esophageal pressure representative signal, and a second sequencer for controlling operation of the second amplifier, second analog-to-digital converter, and second stocking and serializing processor.

31. An EMG_{di} signal and pressure acquisition catheter as defined in claim 28, wherein the first portion of the acquisition and transmission circuit is further supplied with the detected EMG_{di} signal and comprises a selector of the detected gastric pressure representative signal or the detected EMG_{di} signal for being supplied to the amplifier.

32. An EMG_{di} signal and pressure acquisition catheter as defined in claim 30, wherein:
the first memory and serializing circuit produces first serial data supplied to the second memory and serializing circuit;
the second memory and serializing circuit produces second serial data; and
the second portion of the acquisition and transmission circuit further comprises a shaping circuit supplied with the second serial data, the shaping circuit converting the second serial data into a bitstream conforming with a given communication protocol.

33. An EMG_{di} signal and pressure acquisition catheter as defined in claim 32, further comprising a RF data transmitter for transmitting the bitstream from the shaping circuit to a remote processing system.
